(19) United States
(12) Patent Application Publication
(10) Pub. No.: US 2024/0165420 A1
(43) Pub. Date: May 23, 2024

(54) Energy-Based Tissue Treatment and Chilling

(71) Applicant: MiraDry, Inc., San Jose, CA (US)

(72) Inventors: Thomas Pongo, Los Altos, CA (US); Patrick Morin, Hayward, CA (US); Josh Stewart, Fremont, CA (US)

(21) Appl. No.: 18/517,926

(22) Filed: Nov. 22, 2023

Related U.S. Application Data
(60) Provisional application No. 63/427,546, filed on Nov. 23, 2022.

(51) Int. Cl. A61N 5/04; A61N 1/40

(52) U.S. Cl. CPC A61N 5/04; A61N 1/403

(57) ABSTRACT

Embodiments included herein are directed systems and methods for energy-based tissue treatment and chilling. Embodiments of a system may include an energy generator configured to generate a signal. The system may further include at least one antenna configured to receive and transmit the signal. The system may also include a chiller exposed, at least in part, to a housing or cooling fluid that is exposed to a housing of the at least one antenna. The system may additionally include at least one field spreader corresponding to the at least one antenna. Furthermore, the system may include a cooling plate configured to cool an epidermis and a dermis.

### RELATED APPLICATION(S)

This application claims the benefit of U.S. Provisional Application No. 63/427,546, filed on 23 Nov. 2022, the entire contents of which are incorporated herein by reference.

### BACKGROUND

Energy-based therapies such as tissue treatment may be applied to tissue throughout the body to achieve various therapeutic and/or aesthetic results. For example, hyperhidrosis or excessive sweating may be a common condition which can result in excessive sweating in the underarm, facial, back, chest, and/or foot regions of the body. In some cases, hyperhidrosis may be treated using energy-based therapies. The energy-based therapies may provide various degrees of success in treating hyperhidrosis but may also cause adverse side effects including discomfort. It may be desirable to improve energy-based therapies to provide better therapeutic results while limiting adverse side effects such as discomfort.

### SUMMARY OF THE DISCLOSURE

As will be discussed in greater detail below, embodiments of the present disclosure are directed towards systems and methods for energy-based tissue treatment and chilling. Embodiments of a system may include an energy generator configured to generate a signal. The system may further include at least one antenna configured to receive and transmit the signal. The system may also include a chiller exposed, at least in part, to a housing or cooling fluid that is exposed to a housing of the at least one antenna. The system may additionally include at least one field spreader corresponding to the at least one antenna. Furthermore, the system may include a cooling plate configured to cool an epidermis and a dermis.

One or more of the following features may be included. The system may include a thermal structure that surrounds the at least one antenna. The field spreader may be made of ceramic or ceramic filled plastic. The system may further include a switch configured to receive the signal and output the signal to the at least one antenna. The system may also include an applicator. The applicator may be configured to house the at least one antenna, the switch, thermal structure, the at least one field spreader, and the cooling plate. The system may additionally include a console that houses the energy generator. Furthermore, the system may include a power supply configured to power the energy generator, control circuitry for controlling the energy generator, and a display. The energy generator may be a microwave energy generator. The microwave energy generator may be configured to generate a microwave signal having a frequency of about 5.8 GHz. The at least one antenna may be a waveguide antenna. The at least one antenna may be a phased array of four waveguide antennas. The chiller may be a thermo-electric chiller. A cold side of the thermo-electric chiller may touch the housing or fluid that touches the thermal structure surrounding the at least one antenna. The at least one field spreader may be composed of a dielectric ceramic or ceramic filled plastic. The at least one field spreader has a known dielectric constant. The at least one field spreader is a ceramic filled plastic field spreader. A structure made of at least two materials with different dielectric constants, may cause the energy field to spread. A difference between a first dielectric constant corresponding the at least one field spreader, and a second dielectric constant corresponding to a paired field spreader comprising a dielectric ceramic may cause a power density associated with the signal to spread.

In an embodiment, a system for energy-based tissue heating and chilling may include a microwave energy generator configured to generate a microwave signal. The system may further include waveguide antennas configured to receive and transmit the microwave signal. The system may also include a cooling mechanism exposed, at least in part, to a thermal structure that touches a cooling plate. The system may additionally include a first and second field spreader positioned near an end of a waveguide antenna. The system may further include a cooling plate configured to cool an epidermis and a dermis. The first and second field spreaders are dielectric ceramics or ceramic filled plastic and a difference between a first dielectric constant corresponding to the first field spreader and a second dielectric constant corresponding to the second field spreader causes a power distribution associated with the microwave signal to spread.

In an embodiment, a method for energy-based tissue heating and chilling may include generating, by a microwave energy generator, a microwave signal. The method may further include receiving and transmitting the microwave signal with at least one waveguide antenna. The method may also include cooling an epidermis using a thermo-electric chiller exposed, at least in part, to a thermal structure that houses the at least one waveguide antenna and to a cooling plate. The method may additionally include outputting a microwave energy field corresponding to the microwave signal via at least one field spreader positioned near an end of the at least one waveguide antenna. Furthermore, the method may include spreading the microwave energy field using a difference between a first dielectric constant corresponding to the at least one field spreader and a second dielectric constant corresponding to another field spreader. Moreover, the method may include treating the tissue with the microwave energy field.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

This summary is provided to introduce a selection of concepts that are further described below in the detailed description. This summary is not intended to identify essential features of the claimed subject matter, nor is it intended to be used as an aid in limiting the scope of the claimed subject matter.

### BRIEF DESCRIPTION OF THE DRAWINGS

Embodiments of the present disclosure are described with reference to the following figures.

FIG. 1A shows an example device for delivering energy to tissue in accordance with the present disclosure;

FIG. 1B shows an example apparatus for treating tissue with energy in accordance with the present disclosure;

FIG. 1C shows a cross-sectional view of the example device for delivering energy to tissue in accordance with the present disclosure;

FIG. 1D illustrates a time-temperature curve corresponding to treating tissue in accordance with the present disclosure;

FIG. 1E illustrates schematically an underside of an example applicator in accordance with the present disclosure;

FIG. 1F illustrates views of an example applicator with an example handle in accordance with the present disclosure;

FIG. 2 illustrates an example system in accordance with the present disclosure;

FIG. 3 illustrates an example block diagram of a system for energy-based treatment and chilling in accordance with embodiments of the present disclosure;

FIG. 4 illustrates view of an example housing and example antennas in accordance with embodiments of the present disclosure;

FIGS. 5-6 illustrate an example treatment in accordance with embodiments of the present disclosure;

FIG. 7 shows example field spreaders in accordance with embodiments of the present disclosure;

FIGS. 8A-8B illustrate simulations associated with example embodiments of the present disclosure;

FIG. 9 shows an example cooling plate and example microwave connections in accordance with embodiments of the present disclosure;

FIG. 10 shows an example applicator in accordance with embodiments of the present disclosure;

FIG. 11 shows examples of applying microwave energy in accordance with example embodiments of the present disclosure; and

FIG. 12 is a flowchart illustrating an example method for energy-based tissue treatment and chilling according to embodiments of the present disclosure.

Like reference symbols in the various drawings may indicate like elements.

### DETAILED DESCRIPTION

The discussion below is directed to certain implementations. It is to be understood that the discussion below is only for the purpose of enabling a person with ordinary skill in the art to make and use any subject matter defined now or later by the patent “claims” found in any issued patent herein.

It is specifically intended that the claimed combinations of features not be limited to the embodiments and/or implementations and illustrations contained herein, but include modified forms of those implementations including portions of the implementations and combinations of elements of different implementations as come within the scope of the following claims. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure. Nothing in this application is considered critical or essential to the claimed invention unless explicitly indicated as being “critical” or “essential.”

It will also be understood that, although the terms first, second, etc. may be used herein to describe various elements, these elements should not be limited by these terms. These terms are only used to distinguish one element from another. For example, a first object or step could be termed a second object or step, and, similarly, a second object or step could be termed a first object or step, without departing from the scope of the invention. The first object or step, and the second object or step, are both objects or steps, respectively, but they are not to be considered a same object or step.

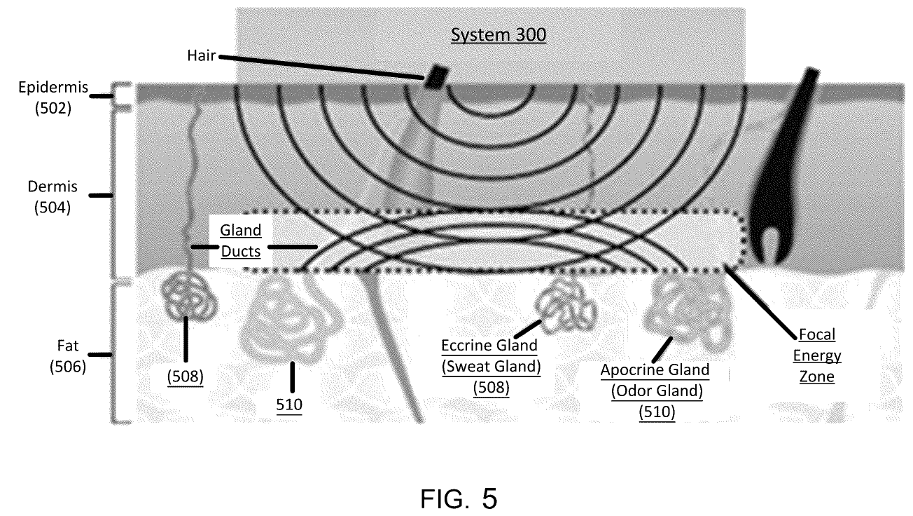

The skin (e.g., of a human) may have three primary layers including the hypodermis, dermis, and epidermis, and may also have internal structures (e.g., as shown in FIG. 5), all of which may be treated. Disclosed herein are methods, apparatuses and systems for non-invasive delivery of energy-based therapy of one or more of these layers or other parts of the body, which in various embodiments may be microwave therapy, based on ceramic based (or waterless) forming of microwave energy for purposes of skin treatment. The energy-based therapy may be delivered to various target tissues to achieve numerous therapeutic and/or aesthetic results. Treatment of target tissue and/or target structures using one or more of the techniques and features described herein may impact the target tissue and/or target structures in one or more of the following ways: modification, deactivation, disablement, denervation, damage, electroporation, apoptosis, necrosis, coagulation, ablation, thermal alteration and destruction. Reaching a temperature in the target tissue and/or target structures therein of at least about 50° C. or more in one or more embodiments of the present disclosure may be used to achieve a desired treatment effect. For example, delivering thermal energy sufficient to heat the target tissue to about 60° C. or more may be used to result in thermal ablation of the target tissue.

It may be desirable to concentrate the treatment within a particular region of dermal and subcutaneous tissue (also referred to herein as the hypodermis) in which target histological structures may reside (e.g., “target tissue”), while doing minimal damage to the tissue above the target tissue in the epidermis and dermis (e.g., “superficial non-target tissue”) and tissue structures within the hypodermis (e.g., “deep non-target tissue”). For example, it may be desirable to treat eccrine glands which may be coiled tubular glands which can be found in the deep dermal layers and/or the upper portion of the hypodermis. Several million glands may generally be present over the surface of the skin, particularly the palms and soles, hairless areas, and axillae. It may also be desirable to treat apocrine glands which may be in, for example, the axilla, perianal and pubic areas, scrotum, labia majora and around the nipples. They may lie generally in the deep dermis and hypodermis and their ducts may terminate in hair follicles.

Depending on the area of the body, a target tissue region may begin anywhere from about 0.5 mm to about 4 mm beneath the skin's surface and end anywhere from about 1 mm to about 10 mm beneath the skin's surface in some embodiments. Further, depending on the area of the body, a superficial non-target tissue region may begin at the skin surface and end anywhere from about 0.5 mm to about 4 mm beneath the skin's surface in some embodiments. Also, depending on the area of the body, a deep non-target tissue region may begin anywhere from about 1 mm to about 10 mm beneath the skin's surface in some embodiments.

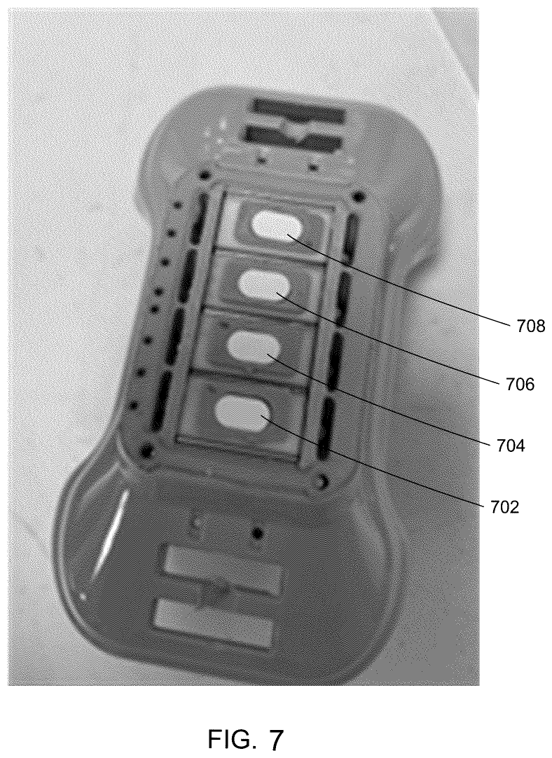

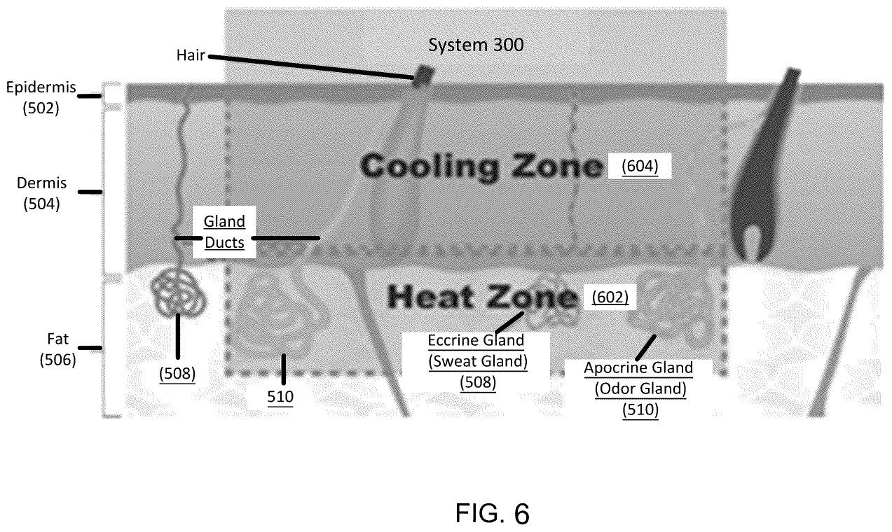

The specific types of tissue structures that may be selected for therapy may depend on the specific therapy or therapies desired. For example, microwave energy as described herein may be delivered to the eccrine or apocrine sweat glands to reduce sweating in a patient (e.g., as shown in FIGS. 5 and 6). Also, apocrine glands may be treated to achieve a reduction in body odor (e.g., as shown in FIGS. 5 and 6). In other examples, microwave therapy as described herein may be used to shrink collagen in the skin for the purposes of skin tightening, wrinkle reduction and/or body sculpting. Microwave therapy may also be used to treat hair follicles, acne, cellulite, vasculature such as varicose veins and telangiectasias, and various other structures. Accordingly, a location of target tissue and non-target tissues may require adjustment based on the specific therapy desired.

Various non-limiting examples of anatomical structures and clinical indications that may be treated by the systems and methods disclosed herein include, but are not limited to, hyperhidrosis (excessive sweating), wrinkles on the skin, bromohidrosis (especially malodorous sweat), chromohidrosis (abnormally colored sweat), acne, cellulite (dimpling of the skin), unwanted hair growth, varicose veins, telangiectasias, benign and malignant skin lesions and infections, and hyperesthesia (e.g., from neurologic disorders). In some embodiments, a plurality of structures/disorders can be treated in the same treatment session.

It should be noted that while microwave energy is discussed herein for treatment of tissue, other energy modalities may be used to achieve the intended therapy. For example, the systems and methods disclosed herein may be configured to deliver one or more of the following modalities: electromagnetic, x-ray, RF, DC, AC, microwave, ultrasound, including high-intensity focused ultrasound (HIFU), radiation, near infrared, infrared, and/or light/laser.

In combination with the thermal treatments disclosed herein, protective treatments may be employed to prevent damage or pain to non-target tissue. For example, thermal protective treatments such as surface cooling may be applied to protect the epidermal layer and portions of the dermal layer of the skin while deeper regions of skin tissue may be heated via energy delivery. Various types of active and passive cooling may be configured to provide this thermal protection to non-target tissue.

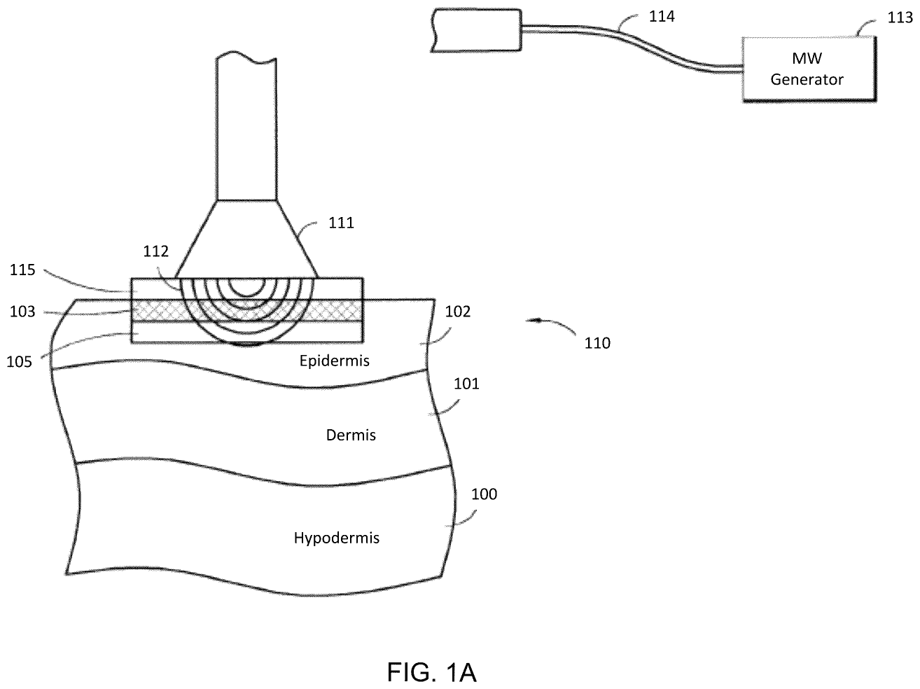

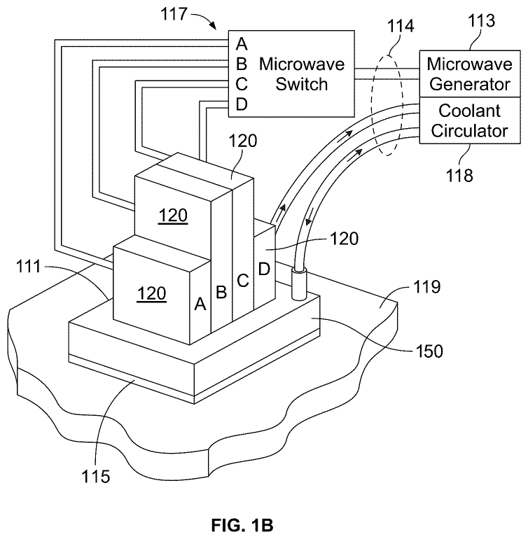

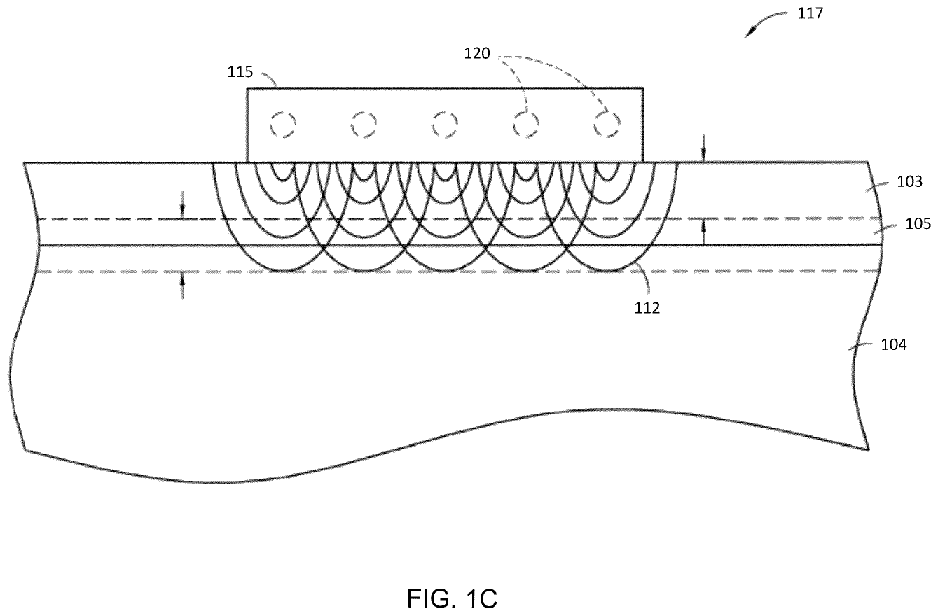

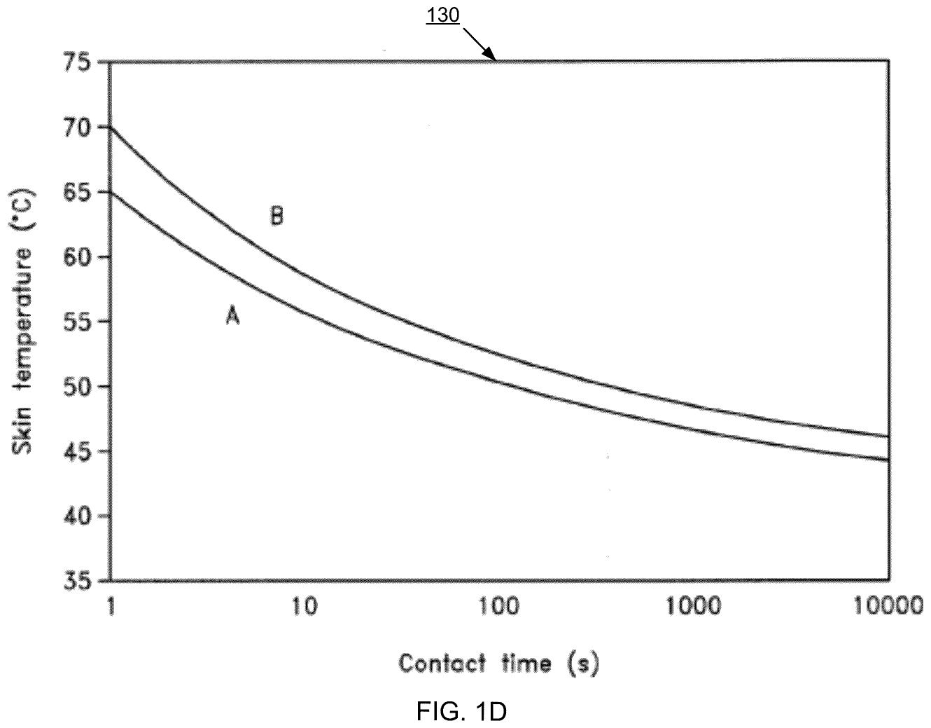

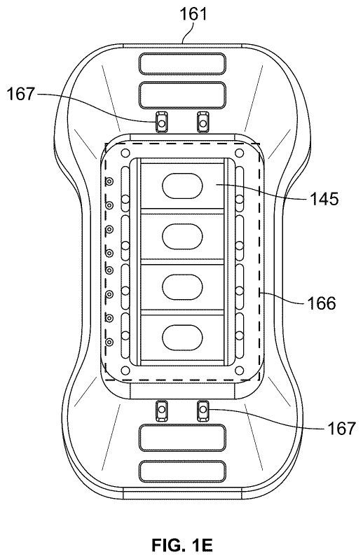

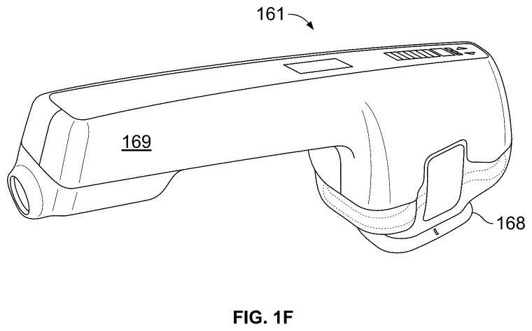

Referring to FIG. 1A, a device 110 having an energy applicator 111 for non-invasively delivering microwave energy 112 to target tissue layer 105 and a microwave generator 113 for supplying the applicator 111 with microwave energy 112 via conduit 114 are shown. The energy applicator 111 may include at least one antenna for delivering microwave energy 112 to the target tissue 105. The antennas may be configured, when the device is placed against or near a patient's skin, to heat and treat the target tissue 105 and target structures within the target tissue 105. The treated target tissue 105 may either be left in place to be resorbed by the body's immune system and wound healing response or be extracted using any number of minimally invasive techniques. Also illustrated is cooling plate 115 for preventing damage to superficial non-target tissue 103.

Microwave energy 112 may be absorbed by the target tissue 105 by a process called dielectric heating. Molecules in the tissue, such as water molecules, may be electric dipoles, wherein they have a positive charge at one end and a negative charge at the other. As the microwave energy 112 induces an alternating electric field, the dipoles may rotate in an attempt to align themselves with the field. This molecular rotation may generate heat as the molecules hit one another and cause additional motion. The heating may be particularly efficient with liquid water molecules, which have a relatively high dipole moment.

Referring to FIG. 1B, an apparatus for treating target tissue with microwave energy may be configured to include the microwave generator 113 connected to the processor, a vacuum system configured to maintain the cooling plate touching the skin, and a device 117 operatively coupled to the generator. The device 117 may further include an energy delivery applicator 111 or energy delivery element such as an antenna for delivering energy to the target tissue. A cable 114 (e.g., feedline) may electrically connect the device to the microwave generator 113. In embodiments, the processor, the device, and/or the microwave generator 113 may be connected wirelessly via, for example, radio frequency signals. The microwave generator 113 may be remotely located from the energy applicator 111, wherein the microwave generator 113 may be either stationary or mobile. Alternatively, the applicator 111 and microwave generator 113 may be coupled such that they comprise a portable unit. Still alternatively, the applicator 111 and microwave generator 113 may be combined into a single unit.

FIG. 1B shows an isometric view depicting an embodiment of a non-invasive energy delivery device 117 comprising multiple microwave antennas 120 that may be electrically connected to the microwave generator 113. The antennas 120 may be contained or housed in a substantially planar applicator plate 115 which may be sized for application against a target area of a patient's skin. The device 117 and the applicator plate 115 therein may be sized and configured to substantially match the area of tissue being treated. Additionally, a vacuum system may be embedded in the energy delivery applicator 111 to maintain contact between the patient's skin and cooling thermally conductive plate (e.g., cooling plate 115).

Referring to FIG. 1C, a cross-sectional side view of the device 117 of FIG. 1B showing delivery of energy 112 into the skin is shown. In such multi-antenna embodiments, it may be useful to orient the antennas 120 along the same plane in the same longitudinal direction to deliver energy in a planar fashion. As shown in FIGS. 1B and 1C (respectively), four or five microwave antennas 120 may be positioned parallel to each other. In other embodiments, fewer or greater microwave antennas 120 may be provided, for example, one, two, three, four, five, six, seven, eight, nine, ten or more. With this planar configuration, energy 112 may be delivered to a larger area of tissue in one treatment and in a more consistent fashion. The antennas may be energized independently, or multiple antennas may be energized at the same time, to apply phase-matched energy delivery to an area that may not be directly under one or more of the antennas.

The amount of energy 112 delivered to the target tissue 105 and consequent extent of treatment effect may be adjusted based on the number of antennas 120, their specific configuration and the power delivered to each antenna 120. For example, a microwave energy output frequency ranging from 300 MHz to 20 GHz may be suitable for feeding the energy delivery device with power. Further, a microwave signal of anywhere from about 915 MHz to about 2450 MHz may yield a treatment effect on tissue. A signal having a frequency ranging from about 2.5 GHz to about 10 GHz may also be useful in some embodiments. Additionally, solid state amplifier, traveling wave tube and/or magnetron components can optionally be used to facilitate the delivery of microwave energy.

In some embodiments, the system may include one or more waveguide antennas which may have a resonant frequency of between about 915 MHz to 15 GHz, and more specifically between about 2.4 GHz to 9.2 GHz, such as about 2.45 GHz and 5.8 GHz in some embodiments. The waveguide antenna(s) may have a cross-sectional size configured to a desired operational frequency and field configuration of the waveguide. Generally, lowest-order Transverse Electric (TE) modes may be utilized (e.g., TE10), although others are possible, such as Transverse Magnetic (TM), evanescent, or a hybrid mode. For example, the width and height (rectangular) or diameter (circular) of the waveguide geometry correlates with the operational frequency and field configuration of the waveguide. Additional parameters, such as fill material, type and placement of feed, and use of mode filtering may affect the operational frequency and field configuration of the waveguide. A transverse mode of a beam of electromagnetic radiation may be a particular intensity pattern of radiation measured in a plane perpendicular (i.e., transverse) to a propagation direction of the beam. Transverse modes may occur in microwaves confined to the waveguide. All modes are time varying.

Transverse modes may occur because of boundary conditions imposed on the wave by the waveguide. Compatible modes may be found by solving Maxwell's equations for boundary conditions of a given waveguide. Transverse modes may be classified into different types. TE modes (Transverse Electric) may have no electric field in the direction of propagation. TM modes (Transverse Magnetic) may have no magnetic field in the direction of propagation. TEM modes (Transverse ElectroMagnetic) may have no electric or magnetic field in the direction of propagation. Hybrid modes may be those which have both electric and magnetic field components in the direction of propagation. An evanescent field may be a time-varying field having an amplitude that decreases monotonically as a function of transverse radial distance from the waveguide, but without an accompanying phase shift. The evanescent field may be coupled, i.e., bound, to an electromagnetic wave or mode propagating inside the waveguide.

The length of the waveguide may be adjusted such that the physical length of the waveguide corresponds to an electrical length that is a half-wavelength multiple of the guided wavelength at the desired operational frequency. This may allow an efficient match from the waveguide feed into the load. Further, the waveguide may have a wide variety of cross-sectional geometries depending on the desired clinical objective and geometry of the particular anatomical area to be treated. In some embodiments, the waveguide has a rectangular, circular, elliptical, or hexagonal cross-sectional geometry. Waveguide applicators may be placed in a phased array configuration for simultaneous or sequential treatment of multiple sites. Additionally, the possibility may exist for beneficial phased (constructive effect of in-phase fields) operation of a waveguide array (similar to the twin coaxial slot antennas).

Additionally, a coaxial feed may be anywhere along the waveguide insertion depth up to the height of the waveguide. The placement may be optimized for efficient transfer of power from coaxial feed to waveguide.

To achieve a desired energy density in a region of the target tissue 105, the antenna 120 may be within 0.5-5 mm of the skin (e.g., between about 1.5-2 mm, such as about 1.75 mm) in some cases, or within several wavelengths of the skin at a given operational frequency in other cases. This distance may be referred to herein as the antenna standoff height. Variation of the standoff height may affect the spread of the microwave radiation. With a very large standoff, a reduced energy density over a larger volume may be achieved. Conversely, with little to no standoff height, the energy density may generally be much higher over a smaller volume. To achieve therapeutic energy density levels with a large standoff, significantly increased input power levels may necessary. The absorption pattern of the microwave energy at depth in tissue, strongly influenced by the standoff, may directly influence the relative safety margin between target 105 and non-target (deep) tissues 104. Finally, standoff height may cause large variation in the loading conditions for the waveguide, with reflected power levels observed by the waveguide antenna changing with standoff changes.

Dielectric filler material may allow waveguides of various cross-sectional area to be utilized and propagated at a specific desired frequency. Cutoff frequency of a fixed size waveguide may be decreased by utilizing larger dielectric constant materials. For example, for a desired treatment size and specified frequency range of 2.4-9.2 GHZ, dielectric filler materials with a dielectric constant of K=2 to 30 may be utilized. Larger K value dielectric filler materials may have a permittivity that is closer to that of tissue, giving the potential for lower reflection in general between the applicator/tissue interface. Some examples of dielectric constants include the skin (K=35-40), fat (K=5-10), muscle (K=50), or water (K=80). In implementations involving a cooling element (e.g., cooling plate 115) or other barrier, the dielectric filler material may be selected based on having a dielectric constant that matches well to the cooling element and skin.

In thermal treatments of tissue, it may be beneficial to protect against the unnecessary and potentially deleterious thermal destruction of non-target tissue. This may particularly be the case in sub-dermal treatments since excess energy delivered to the epidermal 102 and dermal 101 layers of the skin can result in pain, discomfort, drying, charring and edge effects. Moreover, drying, charring and edge effects to surrounding tissue may impair a treatment's efficacy in some cases as the impedance of desiccated tissue may be too high to allow energy to travel into deeper regions of tissue.

To avoid thermal destruction to non-target tissue and any complications associated therewith, an energy delivery device can include a cooling element composed of a thermal mass 150 and cooling plate 115 for providing a cooling effect to the superficial non-target tissue 103 (e.g., the epidermis 102 and portions of the dermis 101). By conductively and/or convectively cooling the epidermis 102 and allowing the cooling effect to penetrate into the dermis 102, the cooling element composed of the thermal mass 150 and cooling plate 115 may establish a zone of thermal protection 103 for the superficial non-target tissue. With the cooling element providing this zone of protection 103, the target tissue 105 may be treated with minimal risk of thermal damage to non-target tissues 103, 104. A method of maintaining contact between the skin and the cooling plate such as a vacuum applied to the treatment zone may be used.

Referring to FIG. 1D, a graph 130 showing a time-temperature curve illustrates a skin temperature above which a burn would be expected (i.e., curve B) and below which no appreciable injury would occur (i.e., curve A) is shown. Accordingly, it may be desirable that, during energy treatment, a cooling system is configured to maintain the non-target skin surface temperature (which can be measured by a temperature sensing element) below curve B for a given treatment duration, as well as below curve A in some cases.

To further reduce the risk of pain and/or other uncomfortable sensations associated with thermal treatment, the cooling element composed of a thermal mass 150 and cooling plate 115 may further cool the superficial non-target tissue 103 to create a numbing effect. Depending on the type of thermal treatment employed and the associated need for complementary cooling, the cooling treatment and resulting cooling and/or numbing effect may be applied before, during, and/or after the thermal treatment. Protective cooling may also be applied in an alternating fashion with the heating treatment to maximize energy delivery while minimizing adverse effects to non-target tissue 103, 104.

The cooling element composed of a thermal mass 150 and cooling plate 115 may take many forms. For example, the cooling element may be a passive heat sink that conductively cools the skin, such as a layer of static, chilled liquid (e.g., water, saline) or a solid coolant (e.g., ice, ceramic plate), a phase change liquid selected which turns into a gas, or some combination thereof (e.g., a cylinder filled with chilled water). The cooling element can also provide active cooling in the form of a spray or stream of gas or liquid, or aerosol particles for convective cooling of the epidermis 102. As will be discussed in further detail below, skin cooling may be provided using a thermal structure 150 that is adjacent to a thermally conductive ceramic plate. The cooling plate is a ceramic plate that touches the patient. The cooling between the thermal structure 150 and cooling plate 115 is conductive. The thermal structure 150 is chilled via contact with a flowing chilled liquid. In some embodiments, a thermo-electric cooler of chiller (TEC) or Peltier element may also be an effective active cooling element. Alternatively, an active cooling element may include a thermally conductive element with an adjacent circulating fluid to carry away heat.

The cooling element may also be incorporated into the device as an internal cooling component for conductively cooling non-target tissue 103, 104. For example, an energy delivery device can couple a cooling component composed of a thermal mass 150 and cooling plate 115 to the energy applicator, where the cooling component plate 115 can passively provide conductive cooling to adjacent tissue. When active cooling is provided, the cooling component composed of a thermal mass 150 and cooling plate 115 may comprise a thermally conductive element 150, wherein a chilled liquid (e.g., water, dry ice, alcohol, anti-freeze) is circulated through the element's internal structure. For example, in microwave energy delivery devices that include a dielectric, the dielectric itself may be a cooling component. In another example, the cooling component plate 115 can be incorporated into the antenna 120 such that it is adjacent to the dielectric.

The cooling component composed of a thermal mass 150 and cooling plate 115 may be incorporated into an energy delivery device 117 comprising at least one microwave antenna 120, such as described above. For example, fluid may be used to cool adjacent skin tissue 119. This type of convective cooling may be enhanced by a coolant circulator 118 that could optionally be integrated within, coupled to, or located remotely from, the microwave generator 113. For example, the cooling circulator 118 may be located remotely from both the microwave generator 113 and cooling plate 115. The properties and characteristics (e.g., medium, flow rate, temperature) of the circulating fluid (gas or liquid) may be selected and modified to achieve the desired cooling effect in light of the amount and rate of energy delivered to the target tissue.

A cooling plate may be thermally conductive and may control a heat transfer rate between tissue and cooling fluid. Further, the cooling plate may be thin (e.g., about 2 mm or less such as 1 mm, 0.75 mm, 0.5 mm, 0.25 mm, 0.20 mm or less in some cases) relative to the wavelength of the microwave signal and may have low electrical conductivity in order to maximize the efficiency of power transfer into the tissue, to keep the waveguide close to the skin and minimize standoff height. Also, the cooling plate may be of adequate stiffness to eliminate bowing while conforming to the skin, thereby maintaining consistent cooling (via constant contact with skin and uniform flow geometries. Additionally, the cooling plate may be made of materials that are transparent to microwave energy (e.g., non-reflective). The cooling plate may be made of any suitable material, for example, glass or a ceramic composite including about 96% alumina, aluminum nitride, or a pyrolytic carbon in some embodiments. Additionally, the cooling plate may have embedded thermocouples to measure cooling plate and skin junction temperature. (Note this is in the “G4” system but the cooling plate is a different material and more thermally conductive, the thermal conductive properties allow for cooling via conduction with a metallic structure instead of direct connection with water).

Low-loss cooling plate materials that meet a permittivity range may be desirable. For example, ceramics such as alumina (K=10), zirconia, silica, aluminum silicate, or magnesia may be used. Further, polymers, such as silicone rubber (K=3), or a ceramic-polymer composite may be utilized. Although specific materials have been described, one skilled in the art will appreciate that the application is not limited to those materials listed. The cooling plate may also be sufficiently thin to minimize undesirable microwave reflection. For example, the cooling plate may be no more than about 10 mm, 9 mm, 8 mm, 7 mm, 6 mm, 5 mm, 4 mm, 3 mm, 2 mm, 1 mm, 0.75 mm, 0.5 mm, or less in thickness.

Controlled delivery of energy may be helpful in avoiding unnecessary damage to target tissue 105 (e.g., desiccation, charring, etc.) and non-target tissue 103, 104 as a result of overheating. A controlled delivery of energy may also result in a more consistent, predictable and efficient overall treatment. Accordingly, it may be beneficial to incorporate into the energy delivery system a controller having programmed instructions for delivering energy to tissue. Additionally, these programmed instructions may comprise an algorithm for automating the controlled delivery of energy. For example, a controller may be incorporated into or coupled to a power generator, wherein the controller may command the power generator in accordance with a preset algorithm comprising temperature and/or power profiles. These profiles may define parameters that can be used in order to achieve the desired treatment effect in the target tissue. These parameters may include, but are not limited to, power and time increments, maximum allowable temperature, and ramp rate (i.e., the rate of temperature/power increase). Feedback signals comprising real-time or delayed physiological and diagnostic measurements may be used to modulate these parameters and the overall delivery of energy. Among the measurements that can be taken, temperature, impedance and/or reflected power at the treatment site and/or target tissue 105 may be particularly useful.

These measurements may help monitor the effect that the energy delivery has at the treatment site and at the target tissue over the course of the treatment. The energy controller may have fixed coefficients or the controller coefficients may be varied depending upon the sensed tissue response to energy delivery. Additionally, an algorithm comprising a safety profile may be employed to limit energy delivery or to limit sensed tissue temperature. These algorithms may shut off energy delivery or modulate the energy delivery. Additionally, in treatments where thermal protection is employed, such as an active cooling system, the protective cooling may be modulated based on the monitored data.

By considering temperature measurements in the delivery of energy, treatment may be administered to achieve the necessary treatment effect while avoiding unnecessary complications of the treatment. For example, energy delivery to target tissue 105 may be steadily increased (i.e., ramped up) until the desired threshold temperature is reached for the target tissue, wherein the threshold temperature is that which is necessary to yield a treatment effect. By ceasing the power increase, or the delivery of energy altogether, once the threshold temperature is reached, harm to non-target tissue 103, 104 resulting from additional and excessive heating may be avoided. In some embodiments, the temperature of the target tissue may be indirectly and noninvasively monitored by determining the temperature of a superficial non-target tissue 103, e.g., at the surface of the skin, and extrapolating from that temperature measurement the target tissue temperature. Adjustments may be made for the skin thickness of a particular patient. For example, it may be desirable to maintain the superficial non-target tissue 103 temperature at less than about 45° C.

Temperature may be measured using any number of sensors, including thermocouples and thermistors, wherein such sensors may be incorporated into the energy delivery element, the energy delivery device and/or the energy delivery system. For example, a thermocouple may be imbedded in the energy applicator, positioned adjacent to the antenna as part of the energy delivery device or located separate from the device such that the thermocouple is wired directly to the generator. The temperature measured may be that of the tissue immediately adjacent the device, the target tissue or any other tissue that may provide useful temperature measurements. In cases where the energy delivery element is in thermal communication with the surrounding tissue (e.g., via conduction), a sensor that is incorporated into the energy delivery element may measure the temperature of the element itself.

Referring to FIG. 1E an underside of the waveguide applicator 161 is illustrated schematically. A waveguide 145, which may be operably connected behind a cooling plate 166 and two vacuum ports 167 each lateral to the cooling plate 166 are shown. Referring to FIG. 1F, on a view of a microwave applicator 161 including a handle 169 for a waveguide antenna system is shown. Also shown, a biotip 168 (e.g., the suction chamber) is shown. The elements shown may preferably be contained within the biotip 168 to facilitate efficient energy delivery, cooling, and suction to a specific area to be treated.

Thermal protective measures may be employed in conjunction with thermal treatments as discussed above. As shown in FIGS. 1B and 1C, the applicator plate 115 containing or housing the antennas 120 may be connected by a conduit 114 to the microwave generator 113, with cooling fluid (e.g., water) passing through the conduit to and from the applicator plate thermal structure 150 from a coolant circulator 118. As shown in FIG. 1A, the cooling fluid may create a protected zone 103 in the epidermis 102 of the patient, so that target tissue 105 below the protected zone is treated. Protected zone 104 is also illustrated in FIG. 1C.

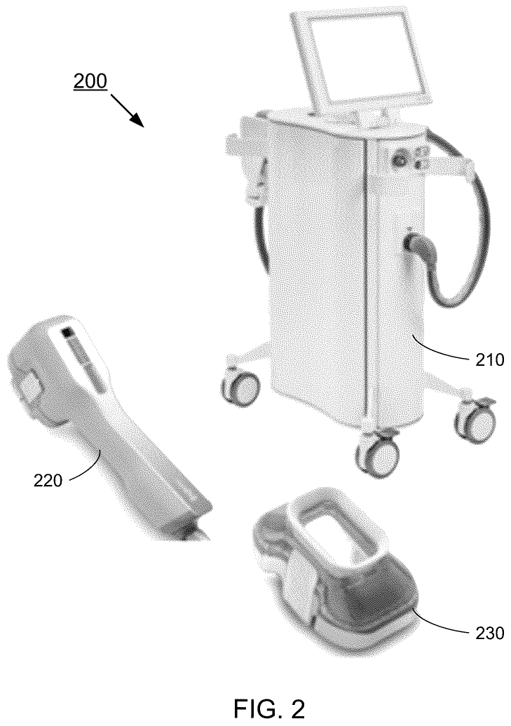

FIG. 2 illustrates an example system 200 in accordance with the present disclosure. The system 200 may be a “G4” system such as those available from the assignee of the present disclosure. The system 200 may use microwaves at, for example, 5.8 GHz to ablate sweat glands. It should be noted that while ablation of sweat glands is described as a capability of the systems and methods discussed herein, this is for illustrative purposes only and other treatments, including but not limited to those referred to herein, are also within scope of capabilities of the systems and methods of the present disclosure.

The system 200 may use a cooling fluid such as water to both cool a top layer of the skin and assist with spreading a microwave field corresponding to the microwaves. The system 200 may include a console 210 (which may house an energy generator such as a microwave energy generator), an applicator 220 (e.g., a handpiece), and a bio-tip 230. The bio-tip 230 may be removably attached to the applicator 220. The bio-tip 230 may couple with or adhere to the applicator 220, as well as to disengage from the applicator 220. Further, the bio-tip 230 may provide a layer between the applicator 220 and a patient using a thin membrane that may allow both microwaves and/or thermal cooling to pass through it. Also, the bio-tip 230 may create a vacuum-tight seal between a treatment site of the patient and a head of the applicator 220. This vacuum capability may be used to pull skin (of the patient) up to make contact with the cooling plate. Additionally, the bio-tip 230 may include a hydrophobic membrane to keep liquids such as blood and lubricant from entering a vacuum tubing of the applicator 220.

Embodiments of the present disclosure are directed towards systems and methods for energy-based tissue treatment and chilling without the use of a cooling fluid (e.g., water), liquid, or the incorporation of a cooling fluid system. For example, the techniques and features described by the present disclosure may be implemented to achieve tissue ablation with energy and with simultaneous liquid-less chilling using a thermo-electric chiller (TEC). In other words, the systems and/or devices described herein may ablate subcutaneous tissue using applied energy while simultaneously cooling a top layer of tissue without use of a liquid coolant.

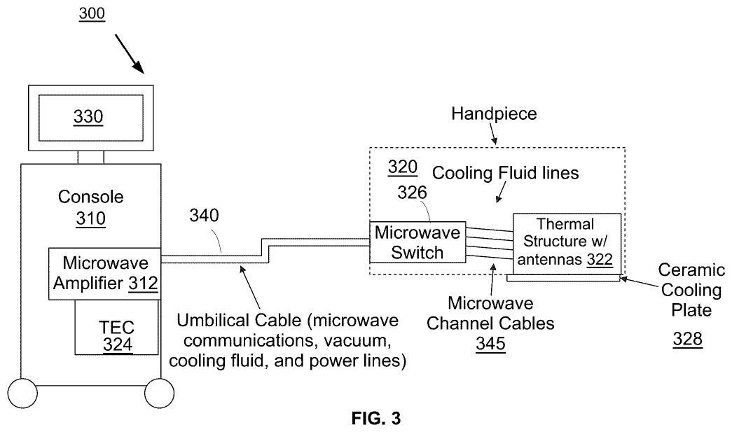

Referring to FIG. 3, an example system 300 in accordance with embodiments of the present disclosure may include a console 310. The console 310 may include or house an energy generator (e.g., a microwave energy generator and/or microwave amplifier 312), a power supply, a controller (e.g., as described above) or control circuitry, and a display 330 (e.g., which may sit on top of, inside of, or exist remotely from, the console 310). The power supply may be configured to power the energy generator (and/or microwave amplifier) 312. Further, the control circuitry (or controller) may be configured to control the energy generator 312 or other components of the system 300. The energy generator (and/or microwave amplifier) 312 may be configured to generate a signal. For example, the energy generator 312 may be a microwave energy generator that may be configured to generate a microwave signal having a frequency of about 5.8 GHZ, although microwave signals generated at other frequencies (e.g., higher and/or lower than 5.8 GHz) by the microwave energy generator are within the scope of the present disclosure.

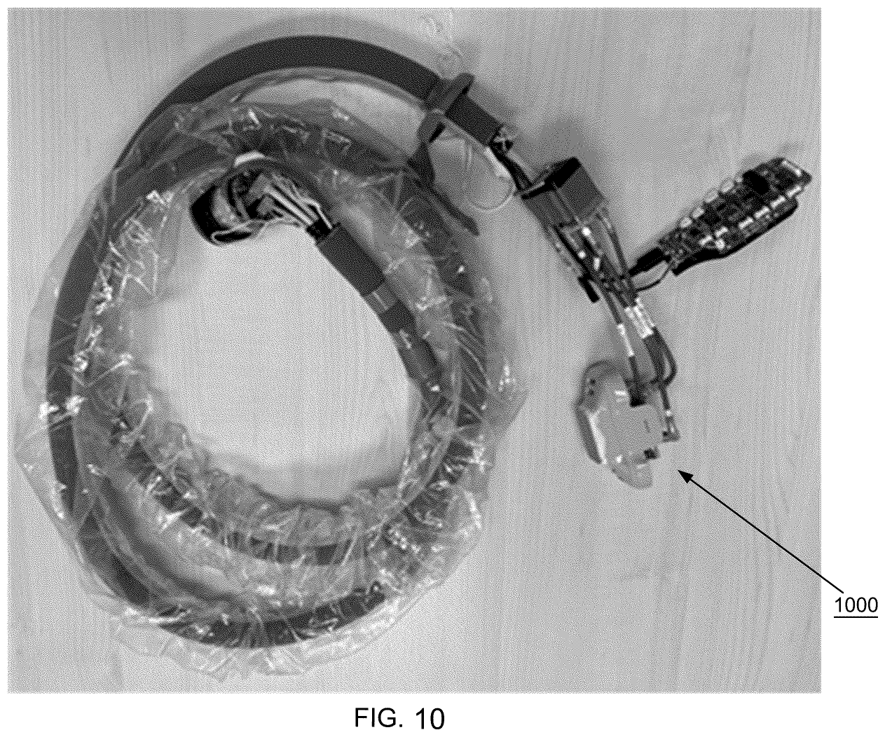

The system 300 may further include (or be configured to include) an applicator 320 (e.g., a handpiece). The applicator 320 may include or house (or be configured to house) one or more antennas for energy application. The one or more antennas may be housed by a thermal structure with antennas 322. The applicator 320 may further include or house (or be configured to house) a TEC 324 which may be used to provide cooling features (e.g., instead of or in place of a liquid or fluid-based cooling system as described above). The system 300 may also include a switch 326 (e.g. a microwave switch) which may be configured to receive the signal (e.g., the microwave signal) generated by the energy generator (and/or microwave amplifier) 312 (e.g. the microwave energy generator) and output the signal to the at least one antenna (e.g., housed by the thermal structure with antennas 322). The switch 326 may be included with or house by the applicator 320. Referring to FIG. 10, an example applicator 1000 in accordance with embodiments of the present disclosure is shown.

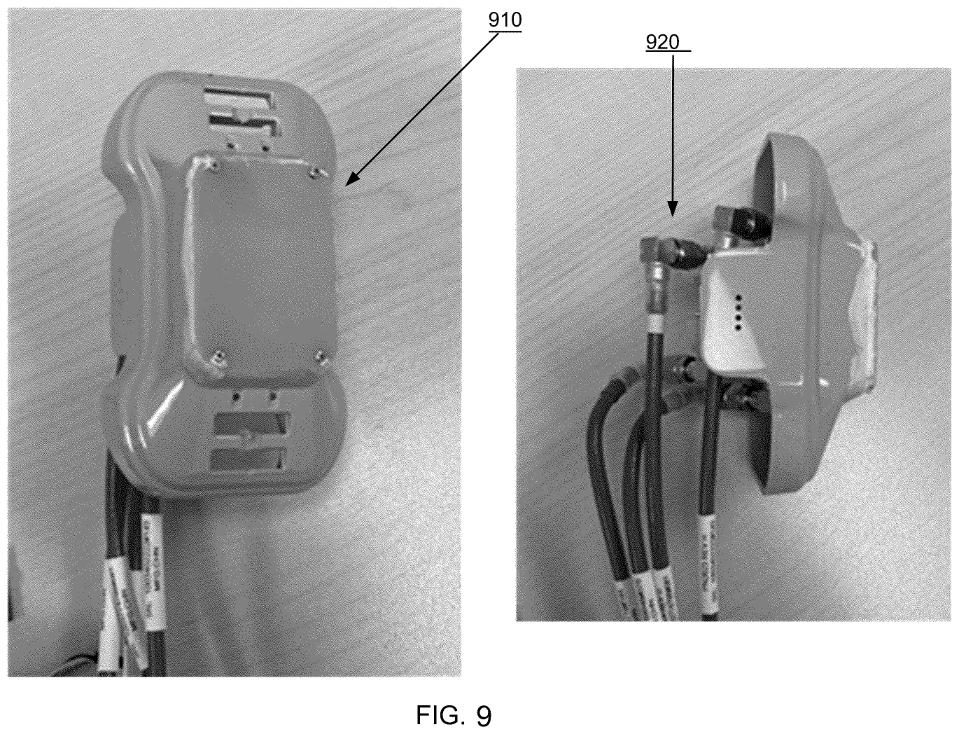

The system 300 may also include cabling 340 (e.g., an umbilical cable) to supply one or more of microwave communications, vacuum, cooling fluid, and/or power from the console 310. Referring to FIG. 9, example microwave connections 920 in accordance with embodiments of the present disclosure are shown. The system 300 may additionally include a bio-tip (e.g., as discussed above with respect to FIG. 2) to provide a barrier between a patient and the system/device (e.g., the applicator 320).

In embodiments, the system 300 may be used for treatment of human tissue and may allow for localization of heating to the skin/fat transition of the dermis without a liquid coolant across the microwave path. The system 300 may additionally include one or more field spreaders (e.g., as part of the applicator 320) that may include or consist of dielectric ceramics, ceramic filled plastic, or air that may spread the energy (e.g., microwave energy) to a desired location/size. The system 300 may be a “G5” system as may become available from the assignee of the present disclosure, Removing the liquid cooling system from the microwave path or capability from, for example, the applicator of prior designs, may also be achieved, at least in part, by loading a ceramic or ceramic filed plastic with a similar dielectric constant to spread the energy/signal (e.g. microwave signal) such that a correct width and/or length of a lesion size is covered by a corresponding energy field.

Further, the system 300 may include at least one antenna that may be configured to receive one or more signals generated by the energy generator 312. The chiller (such as the TEC 324) may be exposed, at least in part, to a housing (e.g., thermal structure with antennas 322) of the at least one antenna. As will be discussed in further detail below, the system 300 may additionally include at least one field spreader corresponding to the at least one antenna.

Also, the system 300 may include a cooling plate 328 (e.g., as part of the applicator 320) configured to cool at least one of an epidermis and a dermis. Referring to FIG. 9, an example cooling plate 910 in accordance with embodiments of the present disclosure is shown. In some embodiments, the cooling plate 910 may be made from a low loss ceramic, which may have a relatively high thermal transfer coefficient (e.g., as compared to alumina, which may also be used). The cooling plate 910 may be in contact with the shroud which, as discussed above, may be cooled by the cold side of the TEC. Further, the material from which the cooling plate is made may allow for microwaves to pass through the cooling place and may also cool the skin.

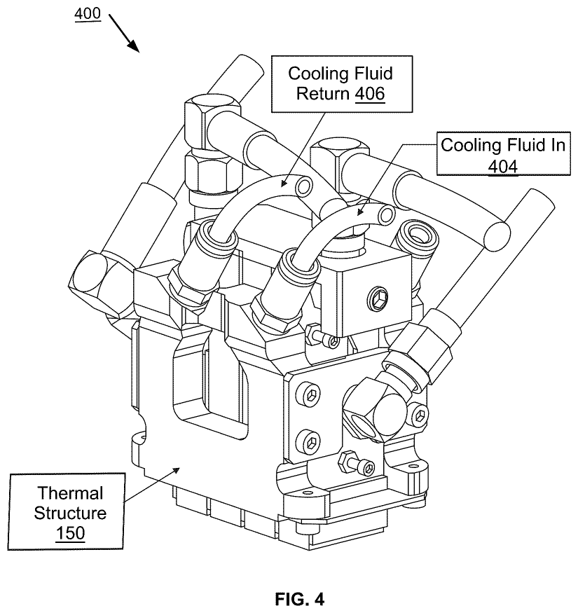

Referring to FIG. 4, an example housing 400 with example antennas in accordance with embodiments of the present disclosure is shown may include a thermal structure 150 (cooling element 150 from FIG. 1B) with a cooling fluid in 404 and a cooling fluid return 406. As discussed, to perform cooling, a TEC may cool a fluid inside the console and pump it to the thermal structure 150 that is surrounding the waveguides as shown in FIG. 4. The thermal structure may be thermally and mechanically attached to the cooling plate and may pull heat away from the patient's skin. The thermal structure may be used to cool the cooling plate used as part of the shaping and shielding of the microwave energy. The cooling fluids return path (cooling fluid return 406) may have cooling fluid that carries away the heat back to the TEC which then cools the fluid again.

Referring to FIGS. 5 and 6, an example treatment in accordance with embodiments of the present disclosure is shown. The example treatment may be for ablation of eccrine and/or apocrine glands. The system (e.g., system 300) may operate by generating a 5.8 GHz microwave signal that may be transmitted via microwave channel cables 345 through an umbilical cable (e.g., cable 340) to a microwave switch (e.g., microwave switch 326). The microwave signal may travel through the microwave switch to be diverted to one or more waveguide antennas (e.g., thermal structures with antennas 322). The microwave signal may travel through one or more of the waveguide antennas and through the output of one or more field spreaders of the antenna (described in further detail below) which may have a different dielectric constant that helps to spread the microwave signal in a more spread-out field pattern. The microwave signal may then travel through the cooling plate (e.g., the cooling plate 328) through the epidermis (502) and the dermis (504) and may be both absorbed and bounced off a fat layer (506) to create a heat zone (602) where the eccrine glands (508) and the apocrine glands (510) may reside. The cooling plate (e.g., the cooling plate 328 as represented near the top of FIGS. 5 and 6 by the reference indicating the system 300) may provide cooling to the epidermis (502) and dermis (504) in, for example, a cooling zone (604) to avoid superficial burns.

Benefits of having a cooling system that does not have cooling fluid going through the microwave path include the option of having various types of fluid as options for cooling fluids. This is because the fluid has dielectric properties that when changed, will also change the effects on the microwave. Another benefit is the system is not influenced by any air bubbles that make its way into the cooling fluid. Using the techniques and features described by the present disclosure for cooling, the cooling zones achieved on the skin may be similar to those achieved for liquid/water cooling that go through the microwave path, though the water-based cooling systems may not have the capability to achieve temperatures below 0 degrees Celsius. This is because water-based systems may freeze at 0 degrees Celsius and systems without water may have temperatures lower than 0 degrees Celsius.

In embodiments, a change in dielectric constants with respect to boundaries may be implemented and a microwave field (e.g., corresponding to the microwave signal discussed above) may be spread out to increase an area that may be treated and to reduce an intensity of the corresponding microwave signal. As discussed above, one or more field spreaders of the systems described herein may be ceramic or ceramic filled plastic, or air. Referring to FIG. 7, example field spreaders 702, 704, 706, and 708 in accordance with embodiments of the present disclosure are shown. One or more of the field spreaders 702, 704, 706, and 708 may be composed of a dielectric ceramic, ceramic filled plastic, or air. Further, ceramic, ceramic filled plastic, or air field spreaders may be implemented at an end of one or more waveguides (e.g., waveguide antennas). One or more of the field spreaders 702, 704, 706, and 708 may have a dielectric constant of about 10. Further, a dielectric ceramic may have a dielectric constant of between 1 to 100.

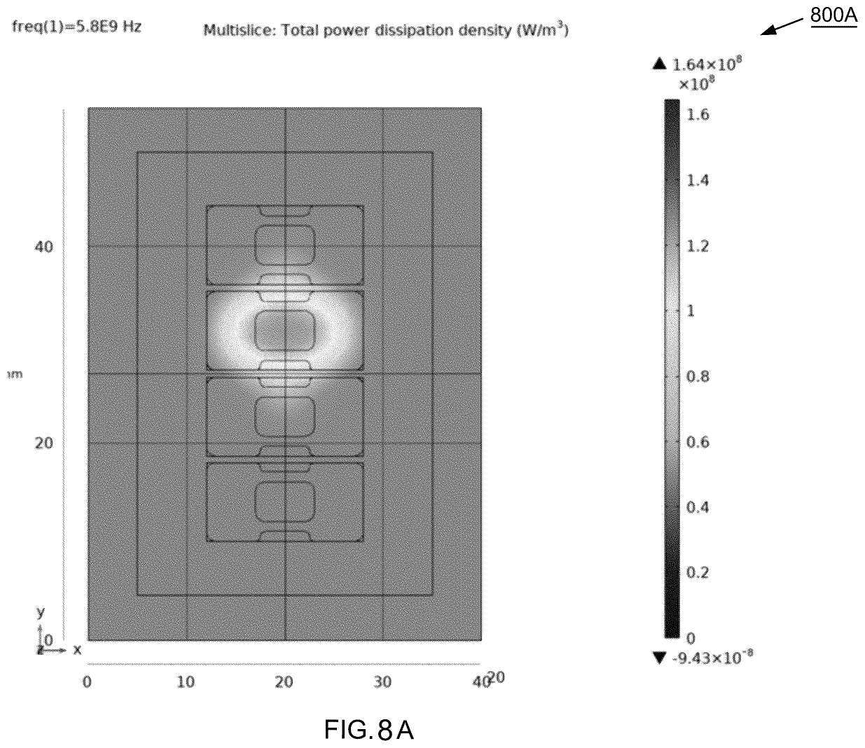

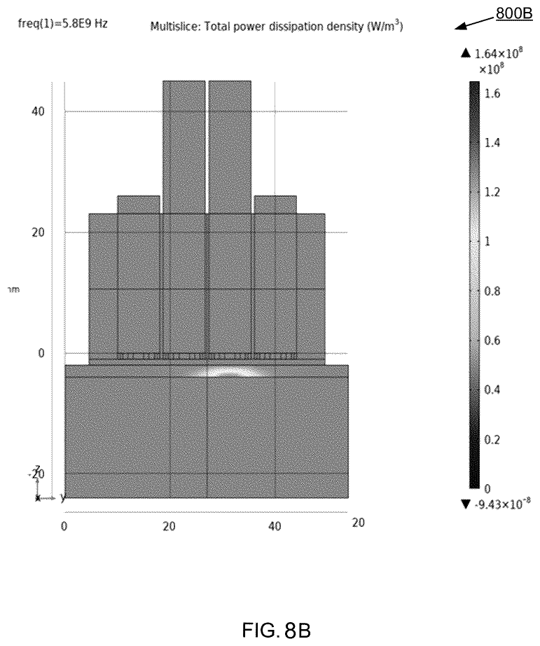

Referring to FIGS. 8A and 8B, graphs 800A and 800B (respectively) illustrating simulations associated with example embodiments of the present disclosure are shown. The simulations may correspond to microwave spreading and localized energy at a skin-fat boundary. The simulations may indicate that when field spreaders with a dielectric constant of 10 are insulated with a dielectric ceramic with a dielectric constant of 100, a spreading of the power density of the microwave signal occurs. Graph 800A of FIG. 8A shows a cross-sectional plot of power dissipation density (heating zones). Graph 800B of FIG. 8B shows a side cross-sectional simulation of power dissipation density with maximum power dissipation (heating) underneath a top layer of the skin (e.g., sub-dermis heating simulated where maximum heating is roughly 2 mm below top layer of skin). The graphs 800A and 800B illustrate that, using the cooling system design describe herein, the power dissipation density outputs may be comparable to cooling systems that use liquid/water that flow through the microwave path.

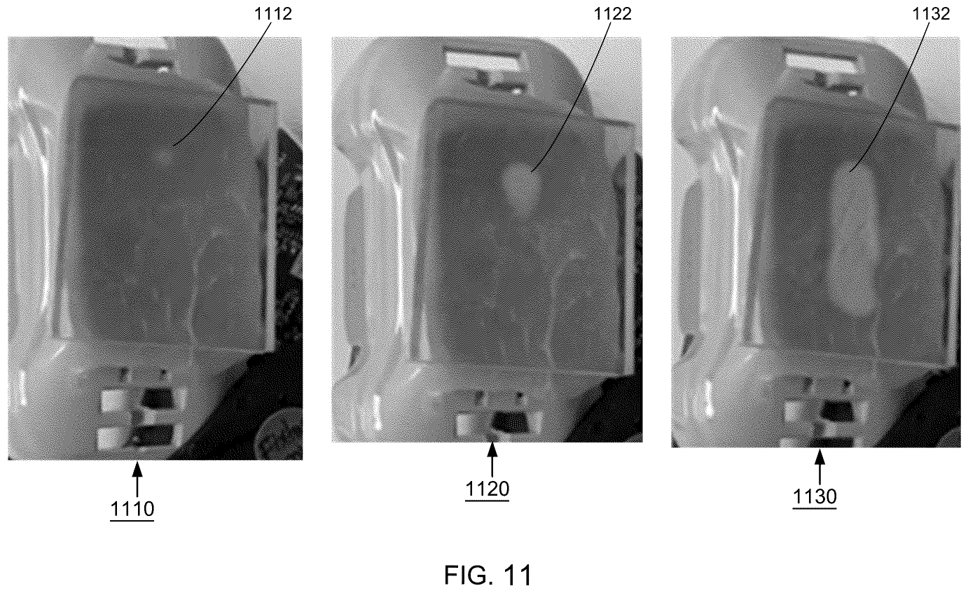

Referring to FIG. 11, examples of applying microwave energy in accordance with example embodiments of the present disclosure are shown. For example, an image 1110 shows a tissue result 1112 after applying microwave energy through a liquid-less (e.g., waterless) microwave field spreading (e.g., as described herein with reference to the system 300) at time T=0 seconds. Further, an image 1120 shows a tissue result 1122 after applying microwave energy through the liquid-less (e.g., waterless) microwave field spreading (e.g., as described herein with reference to the system 300) at time T=3 seconds. Also, an image 1130 shows a tissue result 1132 after applying microwave energy through the liquid-less (e.g., waterless) microwave field spreading (e.g., as described herein with reference to the system 300) at time T=21 seconds. The images of FIG. 11 show tissue (pork in this example) being heated with microwaves from a liquid-less/waterless cooling system design such as those described by the present disclosure. The cooling system used in the examples of FIG. 11 may have seven antennas (e.g., 4 waveguide antennas and 3 phased antennas using two adjacent waveguide antennas at or near the same time) that, when powered on (e.g., one at a time), provide a treatment area similar to the liquid/water-based cooling systems described above. In other words, a phased array of waveguides antennas with field spreading ceramics may shape a microwave signal to treat areas outside of the individual antennas. The images show that the dielectric materials used in place of liquid/water in the cooling system are able to spread out the microwaves to heat an area of tissue similar to the liquid/water-based cooling systems described above.

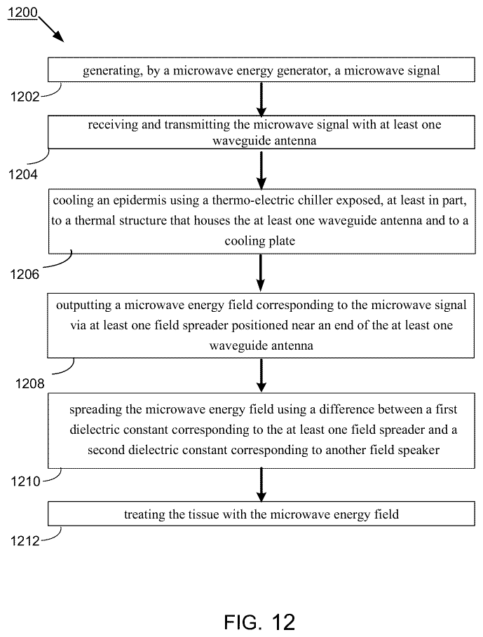

Referring to FIG. 12, a flowchart illustrating an example method or process 1200 for energy-based tissue treatment and chilling according to embodiments of the present disclosure is shown. In an embodiment, a process 1200 for energy-based tissue treatment and chilling may include generating (1202), by a microwave energy generator (e.g., microwave energy generator 312), a microwave signal. The process 1200 may further include receiving (1204) the microwave signal with at least one waveguide antenna (e.g., thermal structure with antennas 322). The process 1200 may also include cooling (1206) an epidermis using a TEC (e.g., TEC 324) exposed, at least in part, to a shroud (e.g., thermal structure with antennas 322) that houses the at least one waveguide antenna and a cooling plate (e.g., cooling plate 328). The process 1200 may additionally include outputting (1208) a microwave energy field corresponding to the microwave signal via at least one field spreader (e.g., one or more of the field spreaders 802, 804, 806, and 808) positioned near an end of the at least one waveguide antenna. Furthermore, the process 1200 may include spreading (1210) the microwave energy field using a difference between a first dielectric constant corresponding the at least one field spreader (e.g., one or more of the field spreaders 802, 804, 806, and 808), Moreover, the process 1200 may include treating (1212) the tissue with the microwave energy field.

Thus, the techniques and features described by the present disclosure may be implemented, as described above in connection with various embodiments of the present disclosure, to reduce or remove complications due to liquid water leaks in tissue treatment systems with liquid cooling systems that may introduce air in the water path. Further, the techniques and features described by the present disclosure may be implemented, as described above in connection with various embodiments of the present disclosure, to reduce or remove the possibility of bio-film build up or debris/contaminants in liquid/water of liquid cooling systems used in tissue treatment systems, that would impede energy delivery or reduce thermal capabilities of the coolant. Also, replacing the water based cooling systems through the microwave path of tissue treatment systems such as those described herein with may simplify manufacturing and/or assembly of the tissue treatment systems and may make the tissue treatment systems easier to maintain and clean, and cheaper to manufacture, assemble, and/or maintain.

It should be noted that while the techniques and features of the present disclosure may be applied to ablate sweat glands, odor glands, and/or hair follicles (e.g., as described above), this is not intended to be a limitation of the present disclosure and other applications of the techniques and features described herein may include, but are not limited to, fat ablation, acne reduction, and other dermal treatments, including but not limited to those discussed herein. Further, it should be noted that the techniques and features described by the present disclosure (e.g., related chillers/field spreaders as described above) may be implemented in a touch-up accessory device (or the like) that may be used to touch-up smaller dermal tissue areas.

The terminology used herein is for the purpose of describing particular embodiments and is not intended to be limiting of the disclosure. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications and variations will be apparent to those of ordinary skill in the art without departing from the scope and spirit of the disclosure. The embodiments were chosen and described in order to best explain the principles of the disclosure and the practical application, and to enable others of ordinary skill in the art to understand the disclosure for various embodiments with various modifications as are suited to the particular use contemplated.

Although a few example embodiments have been described in detail above, those skilled in the art will readily appreciate that many modifications are possible in the example embodiments without materially departing from the scope of the present disclosure, described herein. Accordingly, such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112, paragraph (f) for any limitations of any of the claims herein, except for those in which the claim expressly uses the words ‘means for’ or ‘step for’ together with an associated function.

Having thus described the disclosure of the present application in detail and by reference to embodiments thereof, it will be apparent that modifications and variations are possible without departing from the scope of the disclosure defined in the appended claims.

What is claimed is:

1. A system comprising:
an energy generator configured to generate a signal;
at least one antenna configured to receive and transmit the signal;
a chiller exposed, at least in part, to a housing or cooling fluid that is exposed to a housing of the at least one antenna;
at least one field spreader corresponding to the at least one antenna; and
a cooling plate configured to cool an epidermis and a dermis.

2. The system of claim 1, further comprising:
a thermal structure that surrounds the at least one antenna.

3. The system of claim 1, wherein the field spreader is a ceramic or ceramic filled plastic field spreader.

4. The system of claim 1, further comprising:
a switch configured to receive the signal and output the signal to the at least one antenna.

5. The system of claim 1, further comprising:
an applicator, wherein the applicator is configured to house the at least one antenna, the switch, thermal structure, the at least one field spreader, and the cooling plate.

6. The system of claim 1, further comprising:
a console that houses the energy generator.

7. The system of claim 1, further comprising at least one of:
a power supply configured to power the energy generator;
control circuitry for controlling the energy generator; and
a display.

8. The system of claim 1, wherein the energy generator is a microwave energy generator.

9. The system of claim 8, wherein the microwave energy generator is configured to generate a microwave signal having a frequency of about 5.8 GHz.

10. The system of claim 1, wherein the at least one antenna is a waveguide antenna.

11. The system of claim 1, wherein the at least one antenna is a phased array of four waveguide antennas.

12. The system of claim 1, wherein the chiller is a thermo-electric chiller.

13. The system of claim 12, wherein a cold side of the thermo-electric chiller touches the housing or fluid that touches the thermal structure surrounding the at least one antenna.

14. The system of claim 1, wherein the at least one field spreader is composed of a dielectric ceramic or ceramic filled plastic.

15. The system of claim 1, wherein the at least one field spreader has a known dielectric constant.

16. The system of claim 15, wherein the at least one field spreader is a ceramic filled plastic field spreader.

17. The system of claim 1, wherein a structure made of at least-two materials with different dielectric constants, causes the energy field to spread.

18. The system of claim 14, wherein a difference between a first dielectric constant corresponding the at least one field spreader, and a second dielectric constant corresponding to a paired field spreader comprising a dielectric ceramic, causes a power density associated with the signal to spread.

19. A system for energy-based tissue treatment and chilling, the system comprising:
a microwave energy generator that generates a microwave signal;
waveguide antennas configured to receive and transmit the microwave signal;
a cooling mechanism exposed, at least in part, to a thermal structure that touches a cooling plate;
a first and second field spreader positioned near an end of a waveguide antenna;
a cooling plate configured to cool an epidermis and a dermis; and
wherein the first and second field spreaders are dielectric ceramics or ceramic filled plastic and a difference between a first dielectric constant corresponding to the first field spreader and a second dielectric constant corresponding to the second field spreader causes a power distribution associated with the microwave signal to spread.

20. A method for energy-based tissue treatment and chilling, the method comprising:
generating, by a microwave energy generator, a microwave signal;
receiving and transmitting the microwave signal with at least one waveguide antenna;
cooling an epidermis using a thermo-electric chiller exposed, at least in part, to a thermal structure that houses the at least one waveguide antenna and to a cooling plate;
outputting a microwave energy field corresponding to the microwave signal via at least one field spreader positioned near an end of the at least one waveguide antenna;
spreading the microwave energy field using a difference between a first dielectric constant corresponding to the at least one field spreader and a second dielectric constant corresponding to another field spreader; and
treating the tissue with the microwave energy field.
